(19) United States
(12) Patent Application Publication
(10) Pub. No.: US 2025/0041579 A1
(43) Pub. Date: Feb. 6, 2025

(54) ACCESS DEVICE WITH DUAL ENTRY PORTS

(71) Applicant: ABIOMED, Inc., Danvers, MA (US)

(72) Inventors: Emilia Jahangir, Danvers, MA (US); Evgenij Potapov, Danvers, MA (US)

(73) Assignee: ABIOMED, Inc., Danvers, MA (US)

(21) Appl. No.: 18/789,861

(22) Filed: Jul. 31, 2024

Related U.S. Application Data
(60) Provisional application No. 63/529,851, filed on Jul. 31, 2023.

(51) Int. Cl. A61M 39/06; A61M 39/22

(52) U.S. Cl. CPC A61M 39/06; A61M 39/22

(57) ABSTRACT

Disclosed is an access device for vascular grafts. The access device may allow two large-bore medical devices to be inserted simultaneously. The access device may include a body defining a common arm, a first proximal arm, and a second proximal arm. The common arm may include a common lumen extending from a distal end of the common arm to a proximal end of the common arm. The first arm may include a first lumen extending from a proximal end of the first arm to the common lumen. The second arm may include a second lumen extending from a proximal end of the second arm to the common lumen. The access device may include a plurality of hemostasis valves. A first hemostasis valve may be disposed at the proximal end of the first proximal arm. A second hemostasis valve may be disposed at the proximal end of the second proximal arm.

### CROSS-REFERENCE TO RELATED APPLICATIONS

The present application claims priority to U.S. Provisional Patent Application No. 63/529,851, filed Jul. 31, 2023, the contents of which are incorporated by reference herein in its entirety.

### TECHNICAL FIELD

The present disclosure is drawn to an access device for vascular grafts, and specifically, to an access device for allowing two large-bore devices into a single vascular graft.

### BACKGROUND

Currently no single access device exists to enter two large-bore devices into a single vascular graft. A Y-shaped vascular graft may be used to accomplish this task, which may introduce challenges for medical professionals in use.

### BRIEF SUMMARY

In various aspects, an access device may be provided. The access device may include a body defining a common arm, a first proximal arm, and a second proximal arm. The common arm may include a common lumen extending from a distal end of the common arm to a proximal end of the common arm. The first proximal arm may include a first lumen extending from a proximal end of the first proximal arm to the common lumen. The second proximal arm may include a second lumen extending from a proximal end of the second proximal arm to the common lumen. The access device may include a plurality of hemostasis valves. A first hemostasis valve may be disposed at the proximal end of the first proximal arm. A second hemostasis valve may be disposed at the proximal end of the second proximal arm.

The access device may include a first Touhy-Borst valve coupled to the proximal end of the first proximal arm. The access device may include a second Touhy-Borst valve coupled to the proximal end of the second proximal arm.

The common lumen may have a circular cross-section. The common lumen may have an elliptical cross-section. A first eccentricity of the elliptical cross-section may be less than 0.6.

The distal end of the common arm may be configured to be coupled directly to a vascular graft. The vascular graft may be, e.g., a 10 mm vascular graft. The first lumen may have an inner diameter (including, e.g., an equivalent diameter if the lumen does not have a circular cross-section) of at least 6 mm. The second lumen may have an inner diameter (including, e.g., an equivalent diameter if the lumen does not have a circular cross-section) of at least 6 mm.

An axial length of the common lumen may be greater than or equal to an axial length of the first lumen. An axial length of the common lumen may be shorter than an axial length of the first lumen. A smallest angle A formed between a central axis of the common lumen and a central axis of the first lumen may be 0<A<45°. In some embodiments, 0<A<20°. A smallest angle B formed between a central axis of the common lumen and a central axis of the first lumen may be 0<B<45°. In some embodiments, 0<B<20°.

The access device may include an adapter configured to be removably coupled to the distal end of the body. The adapter may be configured to allow the body to be connected to different sized vascular grafts.

In various aspects, an access device may be provided. The access device may include a body defining a common arm, a first proximal arm, and a second proximal arm. The common arm may have a common lumen extending from a distal end of the common arm to a proximal end of the common arm. The distal end of the common arm may be configured to be attached to a graft. The first proximal arm may have a first lumen extending from a proximal end of the first proximal arm to the common lumen. The second proximal arm may have a second lumen extending from a proximal end of the second proximal arm to the common lumen. The common arm may have a length that is 0.5 to 3 times the length of each proximal arm.

In various aspects, a system may be provided. The system may include a vascular graft operably coupled to a distal end of an access device as disclosed herein.

The system may include a first medical device. The first medical device may be slidably inserted through the first lumen, the common lumen, and the vascular graft. The first medical device may have a maximum outer diameter of at least 6 mm. The first medical device may be a blood pump.

The system may include a second medical device. The second medical device may be slidably inserted through the second lumen and at least partially through the common lumen. The second medical device may have a maximum outer diameter of at least 6 mm. The second medical device may be a cannula.

In various aspects, a kit may be provided. The kit may include an access device as disclosed herein, and one or more adapters configured to be removably coupled to the distal end of the access device.

In various aspects, a method may be provided. The method may include operably coupling a graft to an access device as disclosed herein. That is, the access device may include a body defining a common arm, a first proximal arm, and a second proximal arm. The common lumen may extend from a distal end of the common arm to a proximal end of the common arm. The distal end of the common arm may be configured to be attached to the graft. The first proximal arm may have a first lumen extending from a proximal end of the first proximal arm to the common lumen. The second proximal arm may have a second lumen extending from a proximal end of the second proximal arm to the common lumen.

### BRIEF DESCRIPTION OF DRAWINGS

FIG. 1 is an illustration of an embodiment of an access device.

FIG. 2 is an illustration of a cross-section of an embodiment of an access device.

FIG. 3 is an illustration of an alternative embodiment of an access device.

FIG. 4 is an illustration of an embodiment of a system.

FIGS. 5A-5C are illustrations of embodiments of distal end connections on a common arm for connecting the device to a graft.

### DETAILED DESCRIPTION

The inventors have recognized that there is no single access accessory to enter two large-bore devices into a single vascular graft. In such instances, a Y-shaped vascular graft may be used to accomplish this task. The inventors have further recognized that utilizing a single graft with a single accessory with valves may allow for easier deployment and withdrawal of devices.

Disclosed herein is a dual entry port access device with self-sealing valves, which may join into a single conduit. The two entry ports are intended for simultaneous access of two large bore devices, and the single joint conduit is intended to interface with a vascular graft. The access device may be configured to allow simultaneous entry of two large bore devices (e.g., an IMPELLA® blood pump from Abiomed, Inc., and an extracorporeal membrane oxygenation (ECMO) cannula) into a vascular graft.

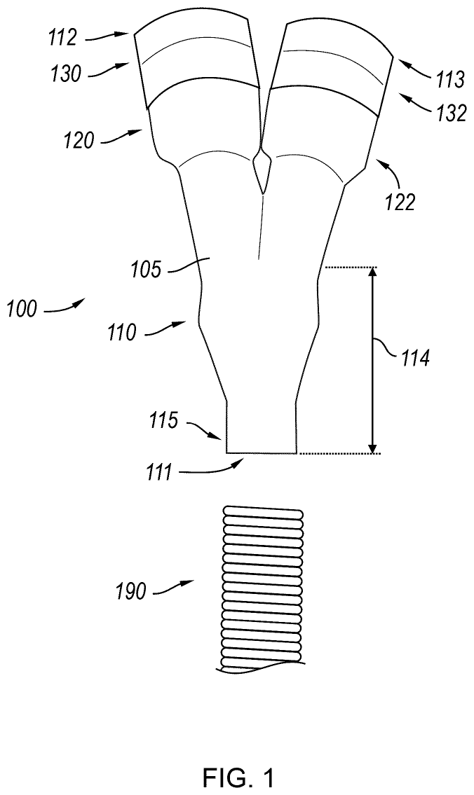

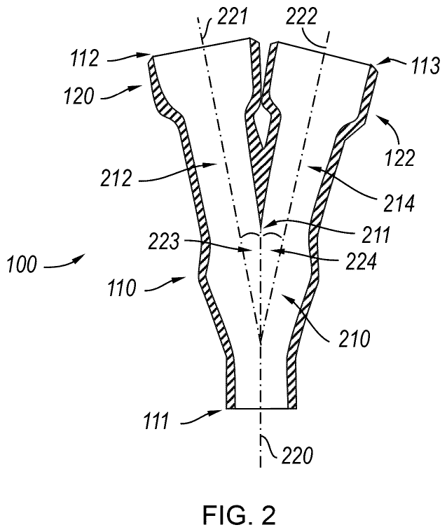

In various aspects, an access device may be provided. Referring to FIG. 1, the access device (100) may include a body (105) defining a common arm (110), a first proximal arm (120), and a second proximal arm (122). Referring briefly to FIG. 2, the common arm (110) may include a common lumen (210) extending from a distal end (111) of the common arm (and the body) to a proximal end (211) of the common arm. The common lumen may have a circular cross-section. The common lumen may have an elliptical cross-section. In some embodiments, the circular shape may have the advantage that it perfectly interfaces with a vascular graft, and, as such, the risk of a leak may be low. In some embodiments, an oval shape may have the advantage that two circular cross-section devices can sit next to each other more easily, therefore allowing for larger devices to enter the accessory.

If the cross-sectional shape is that of an ellipse, then the first eccentricity of that formed ellipse cannot be too large, in order to allow for hemostasis with respect to the graft. In some embodiments, a first eccentricity of the elliptical cross-section may be less than 0.1. In some embodiments, a first eccentricity of the elliptical cross-section may be less than 0.2. In some embodiments, a first eccentricity of the elliptical cross-section may be less than 0.3. In some embodiments, a first eccentricity of the elliptical cross-section may be less than 0.4. In some embodiments, a first eccentricity of the elliptical cross-section may be less than 0.5. In some embodiments, a first eccentricity of the elliptical cross-section may be less than 0.6.

The first proximal arm (120) may include a first lumen (212) extending from a proximal end (112) of the first proximal arm to the common lumen. The second proximal arm (122) may include a second lumen (214) extending from a proximal end (113) of the second proximal arm to the common lumen. The first lumen may have an inner diameter (including, e.g., an equivalent diameter if the lumen does not have a circular cross-section) of at least 5 mm. In some embodiments, the first lumen may have an inner diameter of at least 6 mm. In some embodiments, the first lumen may have an inner diameter of at least 7 mm. The second lumen may have an inner diameter (including, e.g., an equivalent diameter if the lumen does not have a circular cross-section) of at least 5 mm. In some embodiments, the second lumen may have an inner diameter of at least 6 mm. In some embodiments, the second lumen may have an inner diameter of at least 7 mm.

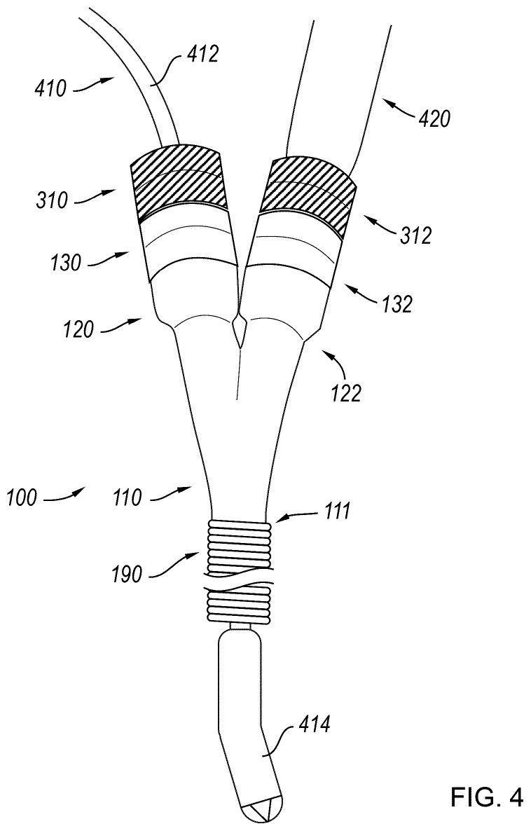

The access device may include a plurality of hemostasis valves. The hemostasis valves may be self-sealing. A first hemostasis valve (130) (see FIG. 4) may be disposed at the proximal end (112) of the first proximal arm. A second hemostasis valve (132) (see FIG. 4) may be disposed at the proximal end (113) of the second proximal arm.

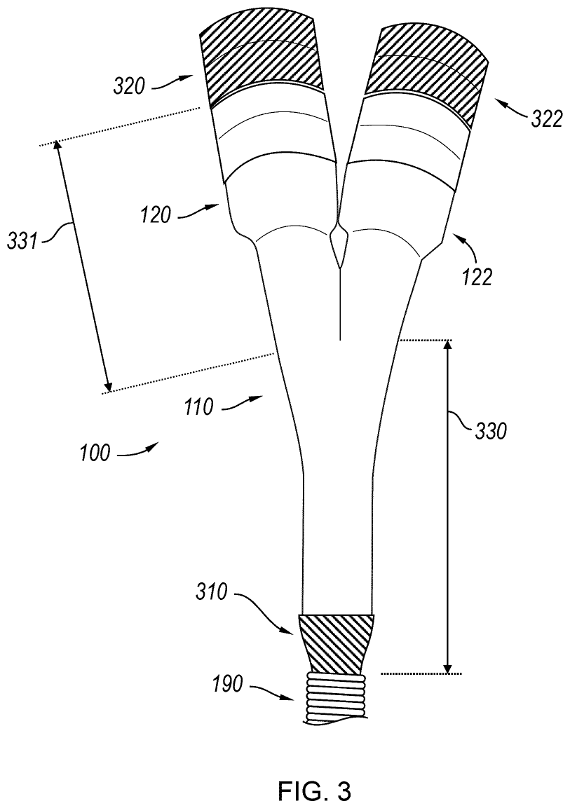

Referring to FIG. 3, the hemostasis valves may include Touhy-Borst valves, with the access device including a first Touhy-Borst valve (320) coupled to the proximal end of the first proximal arm. In some embodiments, the access device may include a second Touhy-Borst valve (322) coupled to the proximal end of the second proximal arm.

The distal end (111) of the common arm (110) may be configured to be coupled directly to a vascular graft (190). Referring to FIG. 3, in some embodiments, the access device may include an adapter (310) at the distal end. The adapter may be configured to be removably coupled to the distal end of the body. The adapter may be configured to allow the body to be connected to different sized vascular grafts. The vascular graft may be any appropriate size. In some embodiments, the vascular graft may be, e.g., a 10 mm vascular graft. As will be appreciated, a clinician may choose the appropriate size of the graft depending upon the patient anatomy and instance of the axial insertion.

In some embodiments, the common lumen may vary in length, depending on the exact application. For example, in some embodiments, an axial length (330) of the common lumen (see FIG. 3) may be greater than or equal to an axial length (331) of the first lumen (e.g., 1.5x longer, 2× longer, 3× longer). In other embodiments, an axial length of the common lumen may be shorter than an axial length of the first lumen (e.g., between 0.5× and 0.75× the length). In some embodiments, the axial length of the common lumen is at least 3 times the length of each of the first and second lumens, individually. In some embodiments, the axial length of the common lumen is at less than 0.5 times the length of each of the first and second lumens, individually. In some embodiments, the axial length of the common lumen is 0.5 to 0.75 times the length of each of the first and second lumens, individually. In some embodiments, the axial length of the common lumen is 0.5 to 1 times the length of each of the first and second lumens, individually. In some embodiments, the axial length of the common lumen is 0.5 to 3 times the length of each of the first and second lumens, individually. In some embodiments, the axial length of the common lumen is 0.75 to 3 times the length of each of the first and second lumens, individually. In some embodiments, the axial length of the common lumen is 1 to 3 times the length of each of the first and second lumens, individually. In some embodiments, the axial length of the common lumen is 1.5 to 3 times the length of each of the first and second lumens, individually.

The common lumen, the first lumen, and the second lumen may be angled relative to each other. Referring to FIG. 2, in some embodiments, a smallest angle (223) (A) formed between a central axis (220) of the common lumen and a central axis (221) of the first lumen may be 0°<A<45°. In some embodiments, 0°<A<20°. A smallest angle (224) (B) formed between a central axis (220) of the common lumen and a central axis (222) of the first lumen may be 0°<B<45°. In some embodiments, 0°<B<20°.

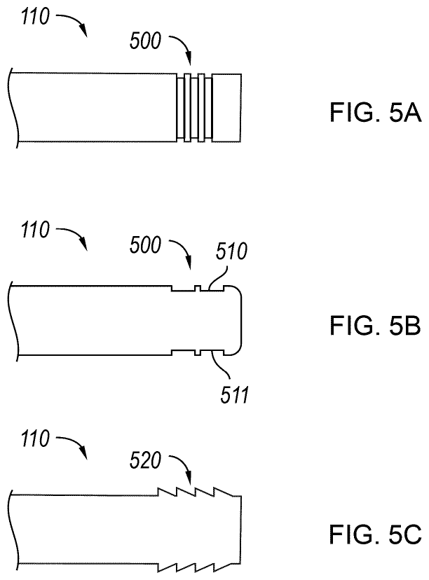

In various aspects, a system may be provided. Referring to FIG. 4, a system may include a vascular graft (190) operably coupled to a distal end (111) of an access device (100) as disclosed herein. For example, in some embodiments, the graft may be sutured onto the access device. In such embodiments, the access device may include a suture ring for connecting the graft to the access device. Referring briefly to FIG. 5A, in other embodiments, the access device may include one or more notches (500), such as a plurality of notches, for connecting the access device to the graft. For example, in such embodiments, a clip may be used to attach the graft to the access device (e.g., via the notches). Note, in FIG. 5A, the notches are shown as a plurality of circumferential channels having a uniform depth extending radially inward from an external surface; in other embodiments, the channels may be formed by protrusions extending radially outward (or a combination thereof). Other notch shapes or designs may be appropriate, including, e.g., referring to FIG. 5B, where each notch may include two or more opposing flat surfaces (510, 511). As will be appreciated, the access device may have other suitable manners for connecting to the graft (e.g., referring to FIG. 5C, a barbed connection (520)). In some embodiments, the system may include a first medical device (410). The first medical device may be slidably inserted through the first lumen, the common lumen, and the vascular graft. The first medical device may have a maximum outer diameter of at least 6 mm. The first medical device may include a blood pump (414). A proximal end of the blood pump may be coupled to a distal end of a catheter (412).

The system may include a second medical device (420). The second medical device may be slidably inserted through the second lumen and at least partially through the common lumen. The second medical device may be slidably inserted at least partially into the vascular graft. The second medical device may have a maximum outer diameter of at least 6 mm. The second medical device may be a cannula.

In various aspects, a kit may be provided. The kit may include an access device as disclosed herein, and one or more adapters configured to be removably coupled to the distal end of the access device.

In various aspects, a method may be provided. The method may include operably coupling a graft (such as a vascular graft) to an access device as disclosed herein. The coupling may occur, e.g., ex vivo or in vivo, as appropriate or desired. In some embodiments, the access device may be directly coupled to the graft. In some embodiments, the access device may be indirectly coupled, via one or more adapters, to the graft.

1. An access device, comprising:
a body defining:
a common arm with a common lumen extending from a distal end of the common arm to a proximal end of the common arm, the distal end of the common arm configured to be attached to a graft;
a first proximal arm with a first lumen extending from a proximal end of the first proximal arm to the common lumen; and
a second proximal arm with a second lumen extending from a proximal end of the second proximal arm to the common lumen; and
a plurality of hemostasis valves including a first hemostasis valve being disposed at the proximal end of the first proximal arm and a second hemostasis valve being disposed at the proximal end of the second proximal arm.

2. The access device of claim 1, further comprising a first Touhy-Borst valve coupled to the proximal end of the first proximal arm.

3. The access device of claim 2, further comprising a second Touhy-Borst valve coupled to the proximal end of the second proximal arm.

4. The access device of claim 1, wherein the common lumen has a circular cross-section.

5. The access device of claim 1, wherein the common lumen has an elliptical cross-section.

6. (canceled)

7. The access device of claim 1, wherein the distal end of the common arm is configured to be coupled directly to a vascular graft.

8. (canceled)

9. The access device of claim 1, wherein the first lumen and the second lumen each have an inner diameter of at least 6 mm.

10. The access device of claim 1, wherein an axial length of the common lumen is greater than or equal to an axial length of the first lumen.

11. The access device of claim 1, wherein an axial length of the common lumen is shorter than an axial length of the first lumen.

12. The access device of claim 1, wherein a smallest angle A formed between a central axis of the common lumen and a central axis of the first lumen is 0<A<45°.

13. (canceled)

14. The access device of claim 1, wherein a smallest angle B formed between a central axis of the common lumen and a central axis of the first lumen is 0<B<45°.

15. (canceled)

16. The access device of claim 1, further comprising an adapter configured to be removably coupled to the distal end of the body.

17. The access device of claim 1, wherein the common arm includes at least one notch on an external surface configured for coupling to the graft.

18. A system, comprising:
an access device of claim 1; and
a vascular graft operably coupled to a distal end of the access device.

19. The system of claim 18, further comprising a first medical device slidably inserted through the first lumen, the common lumen, and the vascular graft.

20-24. (canceled)

25. A kit, comprising:
an access device of claim 1; and
one or more adapters configured to be removably coupled to a distal end of the access device.

26. An access device, comprising:
a body defining:
a common arm with a common lumen extending from a distal end of the common arm to a proximal end of the common arm, the distal end of the common arm configured to be attached to a graft;
a first proximal arm with a first lumen extending from a proximal end of the first proximal arm to the common lumen; and
a second proximal arm with a second lumen extending from a proximal end of the second proximal arm to the common lumen;
wherein the common arm has a length that is 0.5 to 3 times the length of each proximal arm.

27. A system, comprising:
an access device of claim 26; and
a vascular graft operably coupled to a distal end of the access device.

28-33. (canceled)

34. A kit, comprising:
an access device of claim 27; and
one or more adapters configured to be removably coupled to a distal end of the access device.

35. A method, comprising:
operably coupling a graft to an access device, the access device comprising a body defining:
a common arm with a common lumen extending from a distal end of the common arm to a proximal end of the common arm, the distal end of the common arm configured to be attached to a graft;
a first proximal arm with a first lumen extending from a proximal end of the first proximal arm to the common lumen; and
a second proximal arm with a second lumen extending from a proximal end of the second proximal arm to the common lumen.
